(19) United States
(12) Patent Application Publication
(10) Pub. No.: US 2019/0029579 A1
(43) Pub. Date: Jan. 31, 2019

(54) A FLEXIBLE CONTINUOUS PULSE OXYGEN SATURATION MONITOR

(71) Applicants: SHANGHAI MEGAHEALTH TECHNOLOGIES CO., LTD, Shanghai (CN); HANGZHOU MEGASENS TECHNOLOGIES CO., LTD., Hangzhou, ZJ (CN)

(72) Inventor: Congcong ZHOU, Shanghai (CN)

(21) Appl. No.: 16/069,642

(22) PCT Filed: Mar. 25, 2016
(86) PCT No.: PCT/CN2016/077373

§ 371 Date: Jul. 12, 2018

(30) Foreign Application Priority Data
Feb. 6, 2016 (CN) 201610084170.1

(51) Int. Cl. A61B 5/1455; A61B 5/00

(52) U.S. Cl. CPC A61B 5/14552; A61B 5/002; A61B 5/746; A61B 5/6832; A61B 2562/0238; A61B 5/7278; A61B 2560/0214; A61B 2562/164; A61B 5/742

(57) ABSTRACT

A flexible continuous pulse oximeter (6) comprises the following components connected by means of a flexible circuit: a light-emitting diode (LED) array (1, 101, 102, 103) configured to emit testing light; at least one optoelectronic receiving diode (2) configured to receive the testing light and convert the same into an electrical signal; a signal processing unit (3) configured to process the electrical signal and extract a continuous pulse oximetric signal; and power supplies (4, 5) configured to supply power to the entire oximeter.

### TECHNICAL FIELD

A flexible continuous pulse oxygen saturation monitor is disclosed in the present invention, which relates to the field of medical equipment and can be applied to various places where long-time oxygen saturation monitoring needs to be performed.

### BACKGROUND

In recent years, cardiovascular and cerebrovascular diseases are on the rise year by year. The morbidity and mortality of them are at the top of all diseases. There are strong demand for medical equipment that can continuously monitor cardiovascular conditions in community hospitals or family healthcare centers.

The pulse oxygen saturation monitoring apparatus is an apparatus for measuring pulse and oxygen saturation of human body. Oxygen saturation, reflects the oxygen content in the blood, which is critical for the body to maintain metabolism. Effective monitoring of the level of oxygen content in the arteries is an important indicator for determining whether there are disorders in the blood circulatory system and the human respiratory system. Therefore, the monitoring and analysis of pulse oxygen saturation have high medical value and application prospects. There are various oxygen saturation instruments now, but few of them can achieve the demands of comfortable wearing, long-time monitoring of continuous pulse wave oxygen saturation. As biological signals of the body are mostly low frequency weak-signals, these signals must be amplified and post-filtered to meet the requirements for acquisition, and are easily interfered with. The common monitoring apparatus have heavy loads and poor wearing experience, which is difficult to meet the requirements of long-time continuous pulse wave oxygen saturation monitoring.

In order to solve the problems mentioned above, it is essential to design a flexible continuous pulse oxygen saturation monitor.

### SUMMARY

In view of the deficiencies in the prior art, the purpose of the present invention is to provide a flexible continuous pulse oxygen saturation monitor, which has a simple structure, a reasonable design, and is convenient to use. The monitor also has low power consumption and the ability to interact with a remote terminal, it can perform long-time continuous pulse wave oxygen saturation monitoring.

In order to achieve above purposes, the present invention is implemented by the following technical solution: a flexible continuous pulse oxygen saturation monitor, comprising the following connected via a flexible circuit:

a light emitting LED array (1, 101, 102, 103) emitting detection lights;

at least one photoelectric receiving tube (2) for receiving the detection lights and converting them into electrical signals:

a signal processing unit (3) processing the electrical signal and extracting a continuous pulse oxygen saturation signal; and

a power supply (4, 5) supplying power to the whole monitor.

The signal processing unit (3) further comprises a microprocessor control and storage module (301), a wireless transceiver module (302), a signal conditioning module (303), a display module (304), the microprocessor control and storage module (301) connected with the wireless transceiver module (302), the signal conditioning module (303) and the display module (304) respectively: the microprocessor control and storage module (301) is used for local storage; the signal conditioning module (303) processes the electrical signal and extracts a continuous pulse oxygen saturation signal; the wireless transceiver module (302) connects to the remote terminal (7) wirelessly for transmitting alarm information; the display module (304) is used for displaying the continuous pulse oxygen saturation.

Preferably, the automatically calibrated light emitting LED array comprises at least two LED light emitting modules with two different wavelengths, which can adjust light emitting automatically based on the detected signal quality, achieves the function of automatically optimizing alignment, and solves the problem that LED transmitting tube and the receiving tube cannot be directly aligned with each other, which always causes deviation, and affects the measurement accuracy.

Preferably, the signal conditioning module comprises at least two driving circuits for the automatically calibrated light emitting LED arrays, and the driver circuits are multiplex circuits gated using analog switches on which one can be selected from multiple channel, which optimizes the design on the circuit, simplifies the structure, and reduces power consumption.

Preferably, the power supply may be various types of batteries such as a common lithium battery etc., or new energy source (e.g., temperature-difference energy) with energy harvesting apparatus, or flexible battery, which provides the basic guarantee for long-time continuous pulse wave oxygen saturation monitoring in energy supply.

The beneficial effects of the present invention: The apparatus does not require a housing, and can be fixed or self-adhesive to the tissue surface by a user using flexible materials (tapes, finger stalls, etc.) as required. The ultra-low power wireless transceiver module is connected to the remote terminal wirelessly for transmitting alarm information. The alarm threshold can be set by the remote terminal. The wireless module can access to the family healthcare center to complete the alarm, prompt function, and further connect with the community hospital and the central hospital. Therefore, the device is comfortable to wear and can monitor pulse wave oxygen saturation continuously for more than 24 hours, which has strong anti-interference ability and broad application prospects.

### BRIEF DESCRIPTION OF THE DRAWINGS

The present invention is described in detail below with reference to the accompanying drawings and specific embodiments.

FIG. 1 is a schematic structure diagram of the present invention.

FIG. 2 is a schematic diagram of a signal processing unit 3.

### DETAILED DESCRIPTION

In order to make the technical means, creative features, purpose of achievement, and effects of the present invention easy to understand, the present invention is further described in combination with specific embodiments below.

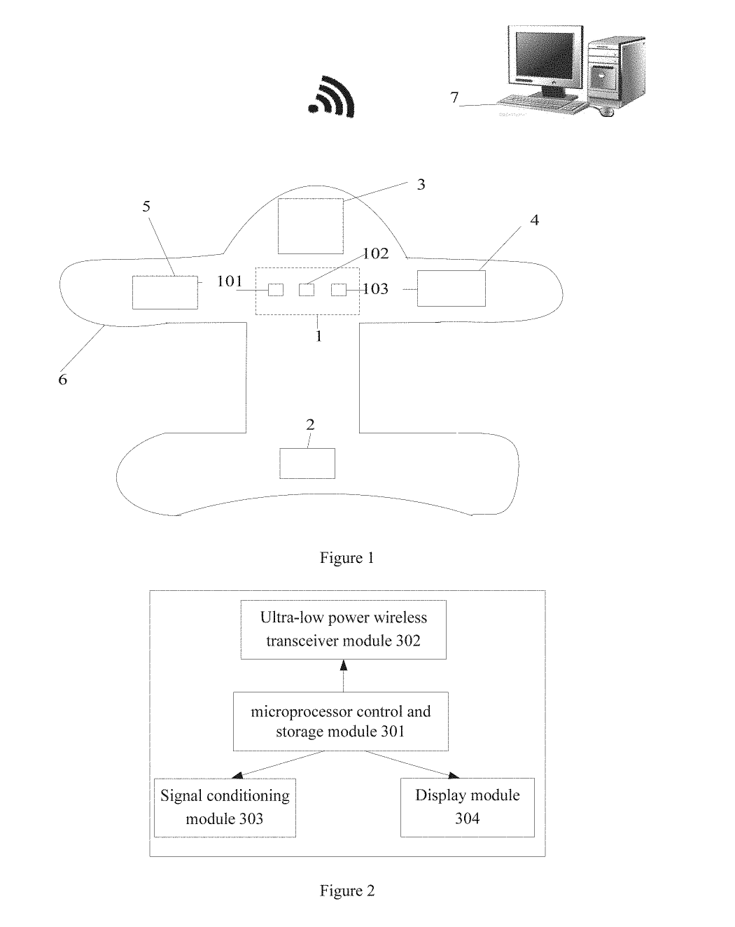

Referring to FIG. 1, the present embodiment adopts the following technical solution: a flexible continuous pulse oxygen saturation monitoring alarm (6) including an automatically calibrated light emitting LED array (1, 101, 102, 103), at least one photoelectric receiver tube (2), a signal processing unit (3), and a power supply (4, 5) connected via a flexible circuit.

The signal processing unit (3), used for processing the electric signal received from the photoelectric receiving tube (2) and extracting a continuous pulse oxygen saturation signal, the structure of it shown in FIG. 2, comprises a microprocessor control and storage module (301), a wireless transceiver module (302), a signal conditioning module (303), and a display module (304). The signal processing unit (3) is connected to at least one photoelectric receiving tube (2) for processing electrical signals. The microprocessor control and storage module (301) is connected with the signal conditioning module (303), the wireless transceiver module (302), and the display module (304) respectively: the microprocessor control and storage module (301) realizes local storage, and the storage time can be adjusted; the wireless transceiver module (302) is connected to the remote terminal (7) wirelessly: the display module (304) is used to display the continuous pulse oxygen saturation; the power supply module (4, 5) supplies power to the whole system and satisfies the function of performing long-time oxygen saturation monitoring.

It is worth noting that the automatically calibrated light emitting LED array (1, 101, 102, 103) for emitting detection lights includes at least two LED light emitting modules with two different wavelengths, LED modules can adjusting light emitting automatically based on the detected signals quality, for example: LED array with two different wavelengths may be bicolor LED of red and infrared, or bicolor LED of green and red. When the design of the entire light emitting row is three bicolor LED light emitting tubes of a first LED light emitting tube 101, a second LED light emitting tube 102, and a third LED light emitting tube 103, the system can automatically adjust the luminous LED according to the quality of signals detected after different people wearing it. The detection method can be polling. At least one photoelectric receiving tube (2) is used to receive the detection lights emitted from the light emitting LED array and convert them into electrical signals.

In this embodiment, the signal conditioning module (303) comprises at least two driving circuits for automatically calibrated light emitting LED arrays (1, 101, 102, 103). The driving circuit is a multiplex circuit gated using analog switches on which one can be selected from multiple channels. After the detection method of polling is completed, the fixed driving circuit needs a connected module.

In the present embodiment, the storage time of the microprocessor control and storage module (301) is adjustable to meet the long-time monitoring function of more than 24 hours. The wireless transceiver module (302) is connected to the remote terminal (7) wirelessly for transmitting alarm information. The alarm threshold may be set by the remote terminal.

The device does not need a structural housing and can be fixed or self-adhesive to the tissue surface by a user using flexible materials (tapes, finger cuffs, etc.) as required. The wireless transceiver module is wirelessly connected to the remote terminal for transmitting alarm information. The alarm threshold may be set by the remote terminal. The wireless module can access to the family healthcare center to complete the alarm, prompt function, and further connect with the community hospital and the central hospital. Therefore, the device is comfortable to wear and can monitor pulse wave oxygen saturation continuously for more than 24 hours, which has strong anti-interference ability and broad application prospects.

The basic principle and main features of the present invention and the advantages of the present invention have been shown and described above. It should be understood by those skilled in the art that the present invention is not limited by the foregoing embodiments. The foregoing embodiments and descriptions only describe the principle of the present invention. There will be various changes and modifications based on the present invention without departing from the spirit and scope of the present invention. Those various changes and modifications are within the scope of the claimed invention. The scope of the invention is defined by the appended claims and their equivalents.

1. A flexible continuous pulse oxygen saturation monitor, comprising the following sections connected via a flexible circuit:
a light emitting LED array comprising a plurality of detection lights;
at least one photoelectric receiving tube capable of receiving light from the detection lights and converting the light into electrical signals;
a signal processing unit capable of processing the electrical signals and extracting a continuous pulse oxygen saturation signal; and
a power supply.

2. The flexible continuous pulse oxygen saturation monitor of claim 1, wherein the signal processing unit further comprises a microprocessor control and storage module, a wireless transceiver module, a signal conditioning module, and a display module,
wherein the microprocessor control and storage module is connected to the wireless transceiver module, the signal conditioning module and the display module respectively;
wherein the wireless transceiver module is capable of connecting to the remote terminal wirelessly for transmitting alarm information;
and wherein the display module is capable of displaying continuous pulse oxygen saturation information derived from the continuous pulse oxygen saturation signal.

3. The flexible continuous pulse oxygen saturation monitor of claim 2, wherein the monitor is fixed or self-adhesive to a tissue surface using a flexible material.

4. The flexible continuous pulse oxygen saturation monitor of claim 2, wherein the plurality of detection lights comprises at least two LED light emitting modules with two different wavelengths.

5. The flexible continuous pulse oxygen saturation monitor of claim 2, wherein the signal conditioning module comprises at least two driving circuits for the light emitting LED arrays and the driver circuits are multiplex circuits gated using analog switches on which one can be selected from multiple channels.

6. The flexible continuous pulse oxygen saturation monitor of claim 2, wherein the wireless transceiver module is capable of transmitting alarm information, and wherein the alarm information is compared to an alarm threshold is set by the remote terminal.

7. The flexible continuous pulse oxygen saturation monitor of claim 2, wherein the storage time of the microprocessor control and storage module is adjustable.

8. The flexible continuous pulse oxygen saturation monitor of claim 2, wherein the power supply is ordinary battery, flexible battery, or new energy source with an energy harvesting apparatus.

9. The flexible continuous pulse oxygen saturation monitor of claim 4, wherein the at least two LED light emitting modules are capable of automatically adjusting the light emitted based on detected signal quality.
